(19) United States
(12) Patent Application Publication
(10) Pub. No.: US 2013/0064772 A1
(43) Pub. Date: Mar. 14, 2013

(54) INFECTION ACTIVATED WOUND CARING COMPOSITIONS AND DEVICES

(71) Applicants: Gerald F. Swiss, Rancho Santa Fe, CA (US); Stefan Schwabe, Palmetto Bay, FL (US); Robert M. Moriarty, Michiana Shores, IN (US)

(72) Inventors: Gerald F. Swiss, Rancho Santa Fe, CA (US); Stefan Schwabe, Palmetto Bay, FL (US); Robert M. Moriarty, Michiana Shores, IN (US)

(73) Assignee: Indicator Systems International, Inc.

(21) Appl. No.: 13/607,431

(22) Filed: Sep. 7, 2012

Related U.S. Application Data
(60) Provisional application No. 61/532,495, filed on Sep. 8, 2011.
(60) Provisional application No. 61/644,969, filed on May 9, 2012.

(51) Int. Cl. A61L 15/44; A61K 49/00

(52) U.S. Cl. CPC A61L 15/44
USPC 424/9.1; 424/447; 514/398; 514/210.09; 514/192; 514/2.3; 514/206; 514/207; 514/253.08; 514/196; 514/29; 514/210.1

(57) ABSTRACT

Provided are wound caring compositions and devices containing a pH-sensitive, preferably acid degradable, components contained in a water-permeable and hydronium ion permeable material. The pH-sensitive component encloses an antibiotic which is released to the wound upon infection by a microorganism at the wound site, and/or encloses a pH indicator. The antibiotic release is triggered by the microorganism's production of CO₂ at the wound site which forms carbonic acid, lowers the pH at the pH sensitive components, and thus results in rupture of the liposome.

### CROSS REFERENCE TO RELATED APPLICATIONS

This application claims the benefit under 35 U.S.C. 119(e) of U.S. Provisional Application Ser. Nos., 61/532,495 filed Sep. 8, 2011, and 61/644,969 filed May 9, 2012, each of which is hereby incorporated by reference into this application in its entirety.

### FIELD OF THE INVENTION

This invention generally relates to compositions and devices suitable for treating wounds, in particular for treating or preventing infections while reducing the risk of drug resistance, and/or for real time detection of incipient infection.

### BACKGROUND OF THE INVENTION

Current antibiotic bandages have an antibiotic ointment on or in the bandage which interfaces the wound. Such a bandage, however, has serious drawbacks. First, an ointment is a homogeneous, viscous, semi-solid preparation, most commonly a greasy, thick oil (e.g., oil 80%-water 20%) with a high viscosity, that is intended for external application to the skin or mucous membranes. Accordingly, the ointment poses a hydrophobic/hydrophilic interface with the wound exudate where bacterial infection is most likely to be present.

Second, the bandage is typically applied immediately after the wound is formed so it acts prophylactically at the wound site. Such a bandage, therefore, can change the endogenous population of bacteria under the bandage by killing off the non-resistant bacteria and leaving a subpopulation of antibiotic resistant bacteria even if an incipient infection is not yet present. When infection occurs, however, the likelihood of an antibiotic resistant infection is increased.

Moreover, premature removal of the bandage as the patient does not detect any infection does not prevent potential, future infection. Finally, an ointment is likely used in these bandages in order to provide a basis to prevent dehydration over time if a water-based antibiotic solution is used.

Furthermore, a real time colorimetric detection of incipient infection at a wound, preferably visibly, can provide early therapy against such infection and provide a faster and better chance of curing that infection.

### SUMMARY OF THE INVENTION

In one aspect, this invention provides topical pH sensitive compositions which comprise an antibiotic, and devices comprising such compositions. Such pH sensitive compositions are stable or substantially stable under basic and neutral pH, preferably under normal physiological pH, but degrade under acidic pH so as to release the antibiotic contained therein. Advantageously, these compositions do not release the antibiotic topically until an actual infection occurs at a topical surface adjacent to or adjoining the composition. Therefore, the compositions of this invention do not unnecessarily interface the antibiotic with the endogenous bacterial population at the wound site thus promoting drug resistance.

In another aspect, this invention provides topical pH sensitive compositions which comprise an antibiotic and a pH indicator, and devices comprising such compositions. Such pH sensitive compositions are also stable under basic and neutral pH, preferably under normal physiological pH, but degrade under acidic pH so as to release the antibiotic contained therein. Advantageously, these compositions also release the antibiotic topically only when an infection occurs at a topical surface adjacent to or adjoining the composition, and additionally also provide a real time, visual detection of the incipient infection.

In the composition and device aspects which include the antibiotic, in some embodiments, “topical” excludes topical administration to oral mucosa.

In yet another aspect, this invention provides topical pH sensitive compositions, which comprise a pH indicator, and devices comprising such compositions.

In some embodiments, the pH sensitive compositions of this invention comprise at least some components which degrade upon a change in pH, such as upon even a slight change of the pH, preferably upon decreasing pH, i.e., upon increasing acidity; the antibiotic and/or the pH indicator (or the “payload”) is contained within such components. In some embodiments, such components are acid degradable components. As used herein, the term “within such components” refers to the payload being included in those components such that, for example, the payload is not substantially released from those components under alkaline or neutral pH; however, the payload is released from those components substantially faster under acidic pH than under alkaline or neutral pH. As is apparent to the skilled artisan, such a release can be easily monitored by assaying the released payload over a range of pH from alkaline to acidic.

In some embodiment, the pH-sensitive components are stable at a neutral or basic pH but degrade at a mildly acidic condition, such as upon contact with a carbonic acid solution formed by incorporation of CO₂ into the water surrounding the components. In an incipient infection, bacteria produce CO₂ and can generate an acidic environment for the components, resulting in rupture, and thus “activation”, of the pH-sensitive, acid degradable, components to release the antibiotic payload.

In some embodiments, such components which degrade upon a change in pH, or preferably, such acid degradable components, can include, without limitation, one or more of pH sensitive, acid degradable, liposomes, micelles, microspheres, nanospheres, matrices, and the like. In some embodiments, the pH sensitive, preferably acid degradable, micelles, microspheres, nanospheres, matrices, and other acid degradable components, comprise one or more pH sensitive, preferably acid degradable, polymers. In some embodiments, the pH sensitive, preferably acid degradable, micelles, microspheres, nanospheres, matrices and other acid degradable components comprise acid degradable hydrogels and xerogels. In some embodiments, the acid degradable hydrogels, and xerogels comprise one or more pH sensitive, or preferably acid degradable, polymers. In some embodiments, acid degradable poly orthoesters (POEs) are not preferred, particularly for use in conjunction with an antibiotic, in the acid degradable components of this invention. In some other embodiments, the pH sensitive composition further comprises an outer layer or membrane, which contains the components that degrade upon a change in pH. A variety of such pH sensitive components are well know in the art.

In various embodiments, the pH sensitive topical compositions comprise a wound dressing such as a bandage, a pad, or a patch. In various embodiments, the wound dressing comprises a cream, a lotion, a liquid bandage, or a film.

Preferably, the payload is immobilized on to the topical composition such that the immobilized payload does not get substantially into systemic circulation and/or the adjoining skin. In one embodiment, such immobilization is provided or enhanced by employing a membrane which is permeable to, for example, water, hydronium ion, and the free antibiotic, but not to the antibiotic immobilized within the pH sensitive component. In one embodiment, the payload is immobilized by anchoring it to a part of the composition or the device of this invention. For example, and without limitation, a liposome containing an antibiotic can have biotin containing lipid molecules and the composition or the device can contain a polymeric material that contains avidin molecules, such that the biotinylated antibiotic containing liposome is immobilized on to the avidin containing material. The pH indicators can be immobilized by covalently attaching the indicator to a polymeric material that is part of the composition or the device. Polymerizable hexa- and heptamethoxy derivatives can be copolymerized with other monomers to form immobilized pH indicators. Such polymerizable pH indicators include those described in U.S. application publication No. 61/570,626, which is incorporated herein in its entirety by reference. The payload can also be immobilized by incorporating it in a matrix, preferably an acid degradable matrix, from which the payload can not leach out or can not substantially leach out under normal physiological pH.

Only when an incipient infection occurs at a wound and releases microbial byproducts which render the aqueous fluid adjacent thereto more acidic, the pH sensitive compositions of this invention release a therapeutically effective amount of the antibiotic. As such, this invention limits antibiotic use on a wound until an infection is present and requires therapeutic intervention.

In the embodiments wherein the composition contains a pH indicator, the indicator is preferably maintained at neutral or slightly basic pH so as to provide for a first color (or no color) at that pH. Upon release into a more acidic environment, the pH indicator changes to another color or becomes colored so as to provide evidence of incipient infection. In a preferred embodiment, the bandage, which comprises the pH indicator, contains the pH indicator in a particular shape such as a +-sign.

In a preferred embodiment, the pH sensitive composition comprising the pH indicator has a clarity such that a change in the indicator color is optically apparent to the viewer.

Preferably, the pH indicators are acid sensitive pH indicators. Such acid sensitive indicators change color when the pH changes, preferably, from neutral or normal physiological to acidic pH. More preferably, the acid sensitive pH indicators are colorless or substantially colorless to the eye at a neutral, basic, or normal physiological pH. Such colorless pH indicators offer an unambiguous way to detect incipient infection at a wound. Even more preferably, the acid sensitive pH indicator that is colorless at neutral or basic pH is hexamethoxy red or heptamethoxy red.

It is also contemplated that certain derivatives of hexa- and heptamethoxy red where one or more methyl groups are replaced with a lipophilic chain like moiety and/or a hydrophilic moiety are also useful in this invention. For example, and without limitation, the derivatives that contain the lipophilic chain like moiety are contemplated to lodge stably within the bilayer membrane of the liposomes, or within the micelles that this invention provides. Such lipophilic chain containing derivatives may also contain one or more hydrophilic moieties as polar head groups that facilitate the inclusion of such derivatives within the liposome's bilayer. The derivatives that contain the hydrophilic moiety are contemplated in certain embodiments to remain in the aqueous part of the liposomes that this invention provides. Accordingly, in some embodiments, the acid sensitive pH indicators useful in this invention are of Formula (I) or a salt thereof:

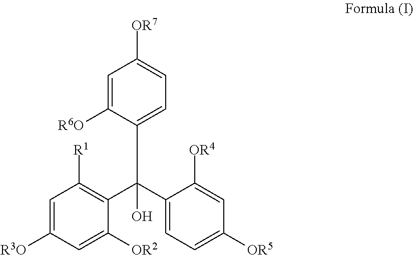

wherein,

R¹ is hydrogen, —OMe, or —OR⁸;

each of R²-R⁸ is independently selected from the group consisting of methyl, -L¹-R⁹, -L²-R¹⁰, a dialkyl glycerol, and a diacyl glycerol;

L¹ is C₄-C₁₈, preferably C₈-C₁₄, alkylene, preferably —(CH₂)ₙ— wherein n is 8 to 14, optionally substituted with 1-8, preferably, 2-6 substituents selected from the group consisting of amino, —CO₂H or an ester thereof, cyano, halo, preferably fluoro, hydroxy, phosphate, and methoxy;

L² is C₁-C₃ alkylene, preferably, C₁-C₂ alkylene optionally substituted with 1-3 substituents selected from the group consisting of hydroxy, phosphate, amino, or CO₂H or an ester thereof;

R⁹ is C₁-C₃ alkyl optionally substituted with 1-5, preferably, 2-3 substituents selected from the group consisting of amino, —CO₂H or an ester thereof, cyano, halo, preferably fluoro, hydroxy, phosphate, and methoxy;

R¹⁰ is amino, —CO₂H or an ester thereof, hydroxy, and phosphate;
phosphate is —OPO(OH)₂— or a mono or di alkyl and/or aryl ester thereof, which ester preferably contains an amino alcohol;

a diacyl glycerol is a moiety of formula —CH₂—C(OCOR¹¹)—CH₂—OCOR¹¹;

a dialkyl glycerol is a moiety of formula —CH₂—C(OR¹¹)—CH₂—OR¹¹; and

R¹¹ is C₈-C₁₈ alkyl or C₈-C₁₈ alkenyl;

provided that at least one of R²-R⁸ is -L¹-R⁸, -L²-R⁹, a dialkyl glycerol, or a diacyl glycerol.

In one aspect, provided herein are methods for assessing incipient infection at a wound employing pH sensitive liposomes comprising long chain fatty acids. In some embodiments, these fatty acids contain up to 25 carbon atoms, and optionally contain up to 4 carbon-carbon double bonds and up to 2 carbon-carbon triple bonds. Non-limiting examples of such fatty acids include stearic acid, oleic acid, palmitic acid, etc. At physiological pH, these acids are primarily in their carboxylate form as shown below:

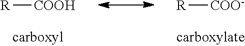

As the pH is lowered due to microbial infection, the amount of the carboxyl form is increased and at some point sufficient numbers of the fatty acid are converted to the carboxyl form so as to disrupt the liposome. The carboxyl group has a —OH absorption band in the infrared spectrum. This band can be measured independent of the liposome disruption to quantify the change in pH and hence the stage of pH change based on incipient microbial growth and microbial infection. As provided herein, this is an alternative to pH indicators as the —OH absorption band of the carboxyl group is readily measured, quantified and correlated to a level of microbial growth and infection.

Extrapolating this to non-liposomal based systems, any component in a wound care device providing a detectable band in the IR that is altered by microbial growth can be used as the basis for IR analysis. For example, a biocompatible polymer can be adjusted to incorporate a certain level of a polymerizable acid functionality such as acrylic acid, methacrylic acid, 4-carboxylstyrene, etc. A scan of the polymer over the wound measuring the OH absorption band is contemplated to simplify the entire process. Using an application to the skin, immediately after surgery, and/or when a wound caring/infection detecting composition is applied on the wound, as the baseline, it is contemplated that the baseline may be subtracted from subsequent readings to accurately determine the of change in pH level.

Aromatic amines and pyridines, are also useful for IR absorption based detection of pH change at a wound site in accordance with various aspects and embodiments of this invention. As used herein, an aromatic amine refers to a molecule containing an amino, alkylamino, or dilakylamino group attached to a aromatic moiety, wherein the aromatic moiety is optionally substituted with 1-3, C₁-C₂₀ alkyl group and/or halo. As used herein, a pyridine refers to a aromatic compound where one CH group is replaced with an —N═ moiety and where the aromatic portion is optionally substituted with 1-3, C₁-C₂₀ alkyl group, halo, and/or C₁-C₆ alkoxy groups.

In certain aspects of this invention are provided methods of detecting presence or absence of an incipient infection at a wound, the method comprising:

(i) contacting the wound with a wound dressing, the wound dressing permitting the accumulation therein of microbial byproducts or derivatives thereof;
(ii) measuring a change of an electromagnetic radiation absorption band of the byproduct or the derivative thereof; and
(iii) correlating the change of the electromagnetic radiation absorption band with the presence or absence of the incipient infection.

As used herein a wide variety of wound dressing well known to the skilled artisan are useful according to this invention. As used herein, “derivative” of microbial byproducts include, without limitation, carbon dioxide and hydrolytic products thereof and reaction products of carbon dioxide and such hydrolytic products with other compounds. Such reaction products include protonated forms of carboxylate anions (or carboxylic acids), protonated amines, such as protonated aromatic amines and pyridines.

In one embodiment, the electromagnetic radiation is infra red (IR) radiation, or ultra violet (UV)-visible radiation. In another embodiment, the wound dressing comprises carboxyl groups. In another embodiment, the IR absorption of carboxyl groups or carboxylate anions corresponding to the carboxyl groups is determined. In another embodiment, the wound dressing comprises a liposome comprising fatty acids, or the composition comprises a poly caroboxylic acid polymer.

Also provided, in other aspects, are device containing the compositions of this invention. In some embodiments, the device includes an outer layer and an inner layer. The inner layer includes a composition as described above. The outer layer, on the other hand, provide support to the inner layer and can be water impermeable and hydronium ion impermeable so that the pH sensitive, and preferably pH degradable, components in the inner layer do not release the antibiotic and/or the pH indicator payload accidentally. The outer layer can optionally include an adhesive surface for adhering the device to a skin.

Also provided are methods of preparing the compositions and devices of this invention as well as uses of such compositions and devices, such as for treating wound infections. In some embodiments the wound infections are caused by one or more of gram-positive cocci, gram negative cocci, gram-negative facultative rods, anaerobes, and fungi. In one embodiment, the gram-positive cocci comprise beta haemolytic Streptococci (such as, Streptococcus pyogenes), Enterococci (such as, Enterococcus faecalis), and Staphylococci (Staphylococcus aureus/MRSA). In another embodiment, the gram-negative rods comprise Pseudomonas aeruginosa. In another embodiment, the gram-negative facultative rods comprise Enterobacter species, Escherichia coli, Klebsiella species, and Proteus species. In another embodiment, the fungi comprise Yeasts (Candida) and Aspergillus.

In one embodiment, the antibiotic useful in this invention is effective against staphylococcus infection. In some embodiments, this invention provides methods for treating a staphylococcus infection at a wound comprising topically administering a pH sensitive, preferably an acid degradable composition of this invention or topically applying a device of this invention comprising a therapeutically effective amount of an antibiotic suitable for treating staphylococcus infection.

In another related aspect, this invention provides a method of measuring the level of an infection at a wound. In some embodiments, the measuring is performed by determining the wavelength of optical absorption and/or the optical density of optical absorption of a wound dressing, which dressing comprises a pH sensitive indicator.

### BRIEF DESCRIPTION OF THE DRAWINGS

FIG. 1 is a side view of one embodiment of the device of this invention.

FIG. 2 shows one embodiment of the device of this invention viewed from the side that is in contact with the wound when in use.

FIG. 3 Schematically illustrates microsphere/nanosphere preparation by oil-in-water (O/W) solvent evaporation technique

### DETAILED DESCRIPTION OF THE INVENTION

Before the compositions and methods are described, it is to be understood that the invention is not limited to the particular methodologies, protocols, assays, and reagents described, as these may vary. It is also to be understood that the terminology used herein is intended to describe particular embodiments of this invention, and is in no way intended to limit the scope of this invention as set forth in the appended claims.

Unless defined otherwise, all technical and scientific terms used herein have the same meanings as commonly understood by one of ordinary skill in the art to which this invention belongs. Although any methods and materials similar or equivalent to those described herein can be used in the practice or testing of this invention, the preferred methods, devices, and materials are now described. All technical and patent publications cited herein are incorporated herein by reference in their entirety. Nothing herein is to be construed as an admission that the invention is not entitled to antedate such disclosure by virtue of prior invention.

When a numerical designation is preceded by the term “about”, it varies by (+) or (−) 10%, 5% or 1%. When “about” is used before an amount, for example, in mg, it indicates that the weight value may vary (+) or (−) 10%, 5% or 1%.

### DEFINITIONS

In accordance with this invention and as used herein, the following terms are defined with the following meanings, unless explicitly stated otherwise.

As used in the specification and claims, the singular form “a”, “an” and “the” include plural references unless the context clearly dictates otherwise. For example, the term “a cell” includes a plurality of cells, including mixtures thereof.

As used herein, the term “comprising” is intended to mean that the compositions and methods include the recited elements, but not excluding others. “Consisting essentially of” when used to define compositions and methods, shall mean excluding other elements of any essential significance to the combination. For example, a composition consisting essentially of the elements as defined herein would not exclude other elements that do not materially affect the basic and novel characteristic(s) of the claimed invention. “Consisting of” shall mean excluding more than trace amount of other ingredients and substantial method steps recited. Embodiments defined by each of these transition terms are within the scope of this invention.

As used herein, Cₓ-C_{y} placed before a group refers to that group including x-y carbon atoms.

As used herein, “alkyl” refers to a monovalent, saturated hydrocarbyl group having 1-20 carbon atoms.

As used herein, “alkenyl” refers to a monovalent hydrocarbyl group having 1-20 carbon atoms and 1-3, carbon-carbon double bonds.

As used herein, “alkylene” refers to —(CR⁹R¹⁰)ₘ— wherein each R⁹ and R¹⁰ independently are C₁-C₃ alkyl, optionally substituted with 1-3 vinylene and/or phenylene groups, optionally substituted with 1-5, preferably 1-3 amino, —CO₂H or an ester thereof, cyano, halo, preferably fluoro, hydroxy, and methoxy, provided that the one or more vinylene groups are only substituted with —CO₂H or an ester thereof, cyano, and halo, preferably fluoro groups.

As used herein, “vinylene” refers to —CH═CH— and “phenylene” refers to divalent 1,2, 1,3, or 1,4 phenyl group.

As used herein, “optionally substituted vinylene” or “optionally substituted phenylene” group substitutes the alkylene group by inserting in between two methylene or substituted methylene units. Non liming and illustrative examples of alkylene groups substituted with 1-3 vinylene and/or phenylene groups include: —(CH₂)₅—CH═CH—(CH₂)₅—; —(CH₂)₅—CH═CH—CH₂—CH═CH—(CH₂)₅—;

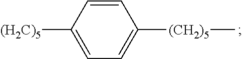

and the likes.

As used herein, “wound dressing” refers to any and all dressings applied over a wound and well known to skilled artisans. Non-limiting examples of wound dressings include, gauze dressings, films, foams, hydrocolloids, alginates, composites, and the like. Gauze dressings include woven or non-woven materials in a wide variety of shapes and sizes. Films, preferably transparent films, include polyurethane material. Foams include film coated gel or a polyurethane material which is hydrophilic in nature. Hydrocolloid dressings can be absorbent and can contain colloidal particles such as methylcellulose, gelatin or pectin that swell into a gel-like mass when they come in contact with wound exudate. Alginate dressings contain salts derived from certain species of brown seaweed. They may be woven or nonwoven and can form a hydrophilic gel when they come in contact with exudate from the wound. In certain preferred embodiments, the wound dressing is a bandage, a pad, or a patch, or a cream, a lotion, a liquid bandage, or a film. In a more preferred embodiment, the wound dressing is a foam.

### Compositions and Devices

This invention provides antibiotic-containing and/or pH indicator, preferably, acid sensitive pH indicator-containing compositions and devices that release the antibiotic to a wound site only upon actual infection at the wound site and/or provide a real time detection of the incipient infection. The incipient infection comprising for example, bacterial, fungal, and/or other microbial growth produce, among others, carbon dioxide, hydrogen sulfide, sulfur dioxide, hydrogen, ammonia, lactate, acetate, formate, citrate, and mixtures thereof. These by-products react with moisture to produce acids such as carbonic acid, sulfurous acid (H₂SO₃), and lactic acid; ammoniam hydroxide; or mixtures thereof, which alter the pH of the immediate environment ultimately reacting with the indicator to produce a color change and/or of an electromagnetic radiation absorption band of the byproduct or derivative thereof.

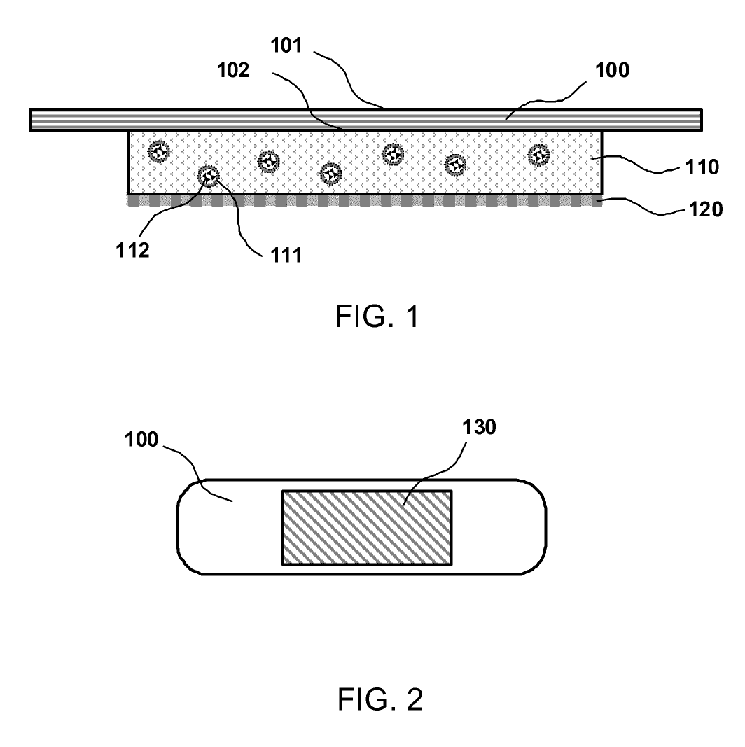

With reference to FIG. 1, one embodiment of this invention provides a composition 110 that is made of a water permeable, hydronium ion permeable and biocompatible material. In one embodiment. entrapped within the material are a plurality of pH-sensitive components 110 optionally enclosing an antibiotic and/or a pH indicator 112.

The material in the composition (110) can include, for instance, woven cotton, woven cellulose, and many other substances known in the art to be water permeable, hydronium ion permeable and biocompatible, such as, for example various polymeric material, hydrogels, and xerogels. For example, water permeable, hydronium ion permeable and biocompatible materials include polymers of 2-hydroxyethyl methacrylate (HEMA), 2-hydroxyethyl acrylate, silicone hydrogels, and the like. In one embodiment, the composition (110) is a foam.

Likewise, the composition can take any shape or size that is suitable for wound caring. In one embodiment, the composition is in the form of a bandage or a pad.

In another embodiment, the composition is in the form of a liquid bandage or a film.

In yet another embodiment, the composition takes a solid form as illustrated as element 110 in FIG. 1. FIG. 1 provides a device contemplated in this disclosure that contains the composition (110), as an inner layer, and an outer layer 100 having a top surface 101 and a bottom surface 102.

In one aspect, the outer layer is water-impermeable. In another aspect, the outer layer is hydronium ion impermeable. When the outer layer is water-impermeable and/or hydronium ion impermeable, it can prevent permeation of acidic solution, of the outside, from accidentally degrading the pH-sensitive components in the composition (110) to release their antibiotic payload.

Materials that can be used to prepare the outer layer include, without limitation, polyethylenes and polypropylenes, both of which are well known in the art and are commercially available.

In some embodiments, the composition (110), in the form of an inner layer, is disposed on the bottom surface (102) of the outer layer (100). Like the outer layer, the inner layer can have a top surface in contact with the outer layer, and a bottom surface that is in contact with the wound when in use.

It is further contemplated that, in one embodiment, either the inner layer contains a top portion that is antibiotic-impermeable, or the outer layer contains a portion that is antibiotic-impermeable. In this respect, accordingly, the inner layer releases the antibiotic to the wound site, without releasing it to the external space.

In some embodiments, the device further includes a skin-contacting surface 120 being a part of the inner layer or as a separate layer. The skin-contacting surface can provide comfort to the skin when the device is applied to the skin. Therefore, in one aspect, the skin-contacting surface is made of a soft and biocompatible material, such as cotton woven and cotton pad, which absorbs water from the wound exudate and thereby forming a water bearing matrix. In another aspect, the skin-contacting surface includes a hydrogel which provides a surface compatible with the skin.

In another aspect, the skin-contacting surface is water and hydronium ion permeable. In another aspect, the skin-contacting surface contains a water soluble antibiotic that is released immediately upon contact with the skin. In yet another aspect, the skin-contacting surface contains an anti-stinging, anti-irritant, or an analgesic compound such as lidocaine. Lidocaine can reduce stinging and burning at the wound site. In one aspect, the skin-contacting surface contains cortisone and/or hydrocortisone to limit inflammation at the wound site.

In some embodiments, as illustrated in FIG. 2, the outer layer (100) extends beyond a wound caring portion 130, which includes the inner layer (110) and optionally the skin-contacting surface (120). In one aspect, the outer layer (100) extends beyond the wound caring portion (130) at at least two directions. In another aspect, the outer layer (100) extends beyond the wound caring portion (130) at all four directions.

In some embodiments, the outer layer (100) further includes an adhesive surface at a portion of the bottom surface of the outer layer that is not covered by the wound caring portion (130). The adhesive surface can be helpful in applying the device to a wound site on the skin.

The compositions and devices of this invention therefore provide a number of advantages over the current technology. First, when an infection does not occur, the antibiotic is not released and the endogenous bacterial population at the wound site is not altered. The compositions and devices of this invention, therefore, are therapeutic and not prophylactic in the sense that they do not cause unnecessary drug resistance. Second, when an infection does occur, such is detected in real time, unambiguously, and optionally, treated simultaneously.

### Antibiotics

Antibiotics are well known in the art and widely available commercially. Any antibiotic can be loaded into the pH sensitive component, with those that are specific to microorganisms that are more commonly involved in wound infections being preferred. In one embodiment, the antibiotic is included within the liposome bilayer, e.g., by being amphoteric or lipophilic, or is incorporated within the aqueous part of the liposome.

Non-limiting examples of antibiotics suitable for use in this invention include adriamycin, amikacin, amphotericin B, ampicillin, azithromycin, bacitracin, benzylpenicillin, bleomycin, capreomycin, carbenicillin, ceftazidime, ceftriaxone, cephalexin, chloramphenicol, ciprofloxacin, clarithromycin, clindamycin, clofazimine, cycloserine, daunorubicin, dibekacin, doxorubicin, doxycycline, enrofloxacin, erythromycin, ethambutol, ethionamide, gentamicin, isoniazid, kanamycin, meropenem, neomycin, netilmicin, oxacillin, paromomycin, penicillin G, piperacillin, polymyxin B, rifabutin, rifampicin, sisomicin, sparfloxacin, streptomycin, teicoplanin, tobramycin, vancomycin and viomycin, and mixtures thereof.

In one embodiment, the antibiotic is selected from the group consisting of ampicillin, ampicillin and sulbactam, augmentin, bacitracin, cefazolin, cefotaxime, cefotetan, cefoxitin, ceftriaxone, cephalexin, ciprofloxacin, dicloxacillin, duricef, erythromycin, imipenem, metronidazole, piperacillin and tazobactam, polymyxin, ticarcillin and clavulanic acid, and combinations thereof.

### pH Indicators

In some embodiments, the inner layer or the skin-contacting surface may also contain a pH indicator for detecting and indicating the presence of bacterial infections. Examples of pH indicators include xylenol blue (p-xylenolsulfonephthalein), bromocresol purple (5′,5″-dibromo-o-cresolsulfonephthalein), bromocresol green (tetrabromo-m-cresolsulfonephthalein), cresol red (o-cresolsulfonephthalein), phenolphthalein, bromothymol blue (3′,3″-dibromothymolsulfonephthalein), p-naphtholbenzein (4-[alpha-(4-hydroxy-1-naphthyl)benzylidene]-1(4H)-naphthalenone), neutral red (3-amino-7-dimethylamino-2-methylphenazine chloride), hexamethoxy red and heptamethoxy red, and combinations thereof. Preferred are pH indicators, that are acid sensitive, i.e., those that change color when the pH is decreased from a normal physiological pH to an acidic pH. More preferred are those acid sensitive pH indicators that are colorless or substantially colorless to a viewer and turn colored, yet more preferably, intensely colored when the pH is decreased. Certain preferred acid sensitive pH indicators include triaryl methane and diaryl methane dyes. The pH indicating moieties in the inner layer or skin-contacting surface are employed in an amount effective for detecting a color change thereby evidencing a change in pH. In a preferred embodiment, the pH indicators exclude base sensitive pH indicators, such as those that are colored under basic pH and colorless under normal physiological pH or under acidic pH. In another embodiment, the base sensitive pH indicators include phthaleins.

In a preferred embodiment, the pH indicators are hexamethoxy red and/or heptamethoxy red or derivatives thereof, such as, for example, compound of Formula (I). These indicators are colorless at a neutral pH (e.g., pH 7.0) and when the pH becomes acidic (e.g., a pH of about 5.0 due to by-products of bacterial growth), the color of the indicator film becomes red. This permits ready determination that bacterial growth has occurred.

Preparation of heptamethoxy red and hexamethoxy red is described in WO2010/085755 which is herein incorporated by reference in its entirety. Compounds of Formula (I) are conveniently prepared based on methods well known to the skilled artisan and commercially available starting material. For example, and without limitation, hexa- or heptamethoxy red is deprotected, preferably under basic conditions, such as using an alkyl or aryl thiolate or PPh₂(−) to provide a triarylmethane compound with one or more phenolic hydroxy groups. Such a compound containing one or more phenolic hydroxy groups are alkylated with X-L¹-R⁸, X-L²-R⁹, an X-dialkyl glycerol, or an X-diacyl glycerol, wherein X is a leaving group, preferably bromo, iodo, or an alkyl or aryl sulfonyloxy (R¹¹—SO₃—) group where R¹¹ is C₁-C₆ alkyl optionally substituted with 1-3 fluoro groups or is phenyl optionally substituted with 1-3 halo or C₁-C₃ alkyl groups. The synthesized compounds are separated by methods well known to a skilled artisan, such as chromatographic separation, recrystallization, or precipitation.

### Acid Degradable Liposomes

In some embodiments, the liposome comprises a water permeable, hydronium ion permeable, and biocompatible material. Further, the liposome is substantially stable at a neutral or basic pH, but at least substantially degrades to release its content at even a mildly acidic condition. Therefore, when applied to a healthy skin or at a wound site that does not have infection, the liposome stays intact and the antibiotic is retained within the liposome.

In some embodiments, the pH sensitive liposomes useful in this invention further comprise cholesterol. Addition of cholesterol to a liposome is contemplated to enhance the liposome's stability without substantially affecting the liposome's pH-sensitive, pH-induced, preferably acid induced degradation.

As used herein, the term “within the liposome” refers to being in the aqueous part inside the liposome and/or being in the bilayered, lipidic, membranous part of the liposome. As will be apparent to the killed artisan in view of this disclosure, a more hydrophobic antibiotic or pH indicator will preferably remain in the bilayered part of the liposome; a more hydrophilic hydrophobic antibiotic or pH indicator will preferably remain in the aqueous part inside the liposome.

As used herein, a “liposome” includes unilamellar and multilamellar liposomes. Unilamellar liposomes have a single spherical bilayer, e.g. that of a phospholipid bilayer, enclosing an aqueous part. These are also referred to as unilamellar vesicles. Multilamellar liposomes have onion-like or multivesicular structures. For an onion-like structure, typically, several unilamellar vesicles form one inside the other in diminishing size, creating a multilamellar structure, e.g., of concentric phospholipid spheres separated by layers of water. Multivesicular liposomes do not have the onion structure, and contains, for example, many smaller non concentric spheres of lipid inside a larger liposome.

pH-sensitive liposomes are known in the art and have been extensively used, for instance, in drug delivery. See review in Drummond et al., “Current status of pH-sensitive liposomes in drug delivery,” Progress in Lipid Research 39:409-60 (2000), which is incorporated herein in its entirety by reference.

In one aspect, the pH-sensitive liposomes of this invention releases their antibiotic and/or pH indicator payload at a pH that is lower than 7. In another aspect, the pH-sensitive liposomes of this invention releases their antibiotic and/or pH indicator payload at a pH that is lower than about 6.9, or 6.8, or 6.7, or 6.6, or 6.5., or 6.4, or 6.3, or 6.2, or 6.1, or 6.0, or 5.9, or 5.8, or 5.7, or 5.6, or 5.5, or 5.4, or 5.3, or 5.2, or 5.1, or 5.0, or 4.5, or 4.0. In yet another aspect, the liposomes can release their antibiotic and/or pH indicator payload at a pH that is higher than about 4.0, or 4.5, or 5.0, or 5.1, or 5.2, or 5.3, or 5.4, or 5.5, or 5.6, or 5.7, or 5.8, or 5.9, or 6.0, or 6.1, or 6.2, or 6.3, or 6.4, or 6.5, or 6.6, or 6.7, or 6.8, or 6.9.

There are at least four types of pH-sensitive liposomes: (A) liposomes that combine polymorphic lipids, such as unsaturated phosphatidylethanolamines, with mildly acidic amphiphiles that act as stabilizers at neutral pH; (B) liposomes composed of “caged” lipid derivatives; (C) liposomes utilizing pH-sensitive peptides or reconstituted fusion proteins to destabilize membranes at low pH; and (D) liposomes using pH-titratable polymers to destabilize membranes following change of the polymer conformation at low pH.

Mildly acidic amphiphiles can be combined with unsaturated phosphatidylethanolamines (PE), such as dioleoylphosphatidylethanolamine (DOPE) to form stable liposomes at neutral pH. N-succinyldioleoylphosphatidylethanolamine (suc-DOPE), oleic acid (OA), palmitoylhomocysteine (PHC), cholesteryl hemisuccinate (CHEMS), and 1,2-dioleoyl- or 1,2-dipalmitoyl-sn-3-succinylglycerol (DOSG or DPSG) are a few of the stabilizers that have been used to prepare pH-sensitive liposome formulations. A most common feature of these lipids is the net negative charge at neutral pH that allows it to stabilize DOPE-containing membranes. Liposomes composed of these lipids can stably encapsulate highly water-soluble drugs, including peptides, at neutral pH. However, in a mildly acidic environment the stabilizer becomes protonated, resulting in membrane destabilization and degradation.

“Caged” liposomes refer to liposomes that reversibly express a particular property, which may include the ability to form fusion competent non-bilayer phases or more simply drug permeable membranes. “Caged” liposomes can be prepared with pH-labile N-maleylphosphatidylethanolamine derivatives. The “caging” process has involved both the reversible covalent modification of a nucleophilic functionality on the lipid head group or cleavage of an alkyl group, releasing membrane destabilizing fatty acids and lysolipids. The synthesis of pH-labile N-maleyl DOPE derivatives have been described, that release the stabilizing group at low pH and thus simultaneously increase the concentration of the destabilizing component, DOPE, and decrease the concentration of the stabilizing component, N-citra-conyl-DOPE.

pH-sensitive peptides used to destabilize liposome membranes include, for instance, GALA, SFP, Poly(Glu-Aib(2-aminoisobutyric acid)-Leu-Aib), EGLA-I, EGLA-II, JTS1, Rhinovirus VP-1, INF3, INF5, INF7, INF8, INF9, INF10, HA peptide, D4, E5, ESL, E5NN, E5CC, ESP, E5CN, and AcE4K.

Synthetic polymers can be used to make pH-sensitive liposomes. Surface-active polymers are capable of sensitizing phospholipid bilayer membranes to a variety of environmental stimuli such pH, temperature or light. This approach appears as a promising alternative to PE-based formulations and pH-sensitive fusogenic peptides for the preparation of pH-sensitive liposomes. Synthetic polymers present several favorable characteristics including low immunogenicity, straightforward large-scale synthesis, structure versatility and easy association to the liposome surface. Furthermore, pH-responsive polymers can be used to prepare pH-sensitive liposomes of almost any composition.

Acid-triggered liposome destabilization/fusion is generally achieved by using non-peptidic polyelectrolytes. Acid tritration of the polymer is usually accompanied by a modification of the polymer conformation and/or association with the liposome bilayer which results in its destabilization. The mechanism of membrane destabilization varies depending on whether the polyelectrolyte is a weak base (polycation) or a weak acid (polyanion).

Polymers that have pH-dependent fusogenic properties include synthetic polypeptides such as poly(1-lysine) or poly(1-histidine). At high pH values, these polymers are neutral but acquire a positive charge as the pH decreases. In solution, the ionized polymer can interact with negatively charged membranes, perturb lipid packing and promote aggregation and fusion of liposomes.

Weak acid polyelectrolytes differ from polycations in that they can trigger contents leakage from neutral as well charged liposomes. One common characteristic of all pH-sensitive polyanions described to date is that they bear carboxylic acid groups which state of ionization determines the polymer's ability to interact/destabilize lipid bilayers. Non-limiting examples include poly(acrylic acid) derivatives, succinylated poly(glycidol)s of and copolymers of N-isopropylacrylamide (NIPAM).

The liposomes useful in this invention are prepared following methods well known to a skilled artisan such as hand-shaken vesicles method (or thin-film method), sonicated vesicles methods, freeze-dried rehydration vesicles method, reverse phase evaporation method, large unilamellar vesicles by extrusion technology, and dehydration rehydration vesicle method. The liposomes useful in this invention are separated using various methods well known to the skilled artisan such as size exclusion chromatography, centrifugation, and the likes. The liposomes useful in this invention are characterized by methods well known to the skilled artisan. For example, the lamellarity of the liposomes is measured by measuring the average particle diameter, or using electron microscopy or cryo electron microscopy. The size of liposomes is measured by electron microscopy or by laser light scattering.

### Acid Degradable Micelles

In some embodiments, the component which degrades upon a change in pH is a micelle. In some embodiments, the micelle comprises one or more polymers, preferably acid degradable polymers. See, for example, Yuan et al., Nanotechnology. 2011, 22(33):335601 and Tang et al., J. Control Release. 2011, 151(1):18-27 (each of which is incorporated herein in its entirety by reference).

An exemplary and non-limiting acid degradable micelle is prepared as follows. A polyethylene glycol detachable graft copolymer, mPEG-g-p(NAS-co-BMA), is synthesized by grafting 2-(ω-methoxy)PEGy1-1,3-dioxan-5-ylamine onto poly(N-(acryloyloxy)succinimide-co-butyl methacrylate). Pseudo in situ cross-linking of the mPEG-g-p(NAS-co-BMA) is performed in dimethyl formamide phosphate buffer (v/v=1/1) by an acid-labile diamine cross-linker bearing two symmetrical cyclic orthoesters. The cross-linked (CL) micelles with different contents of mPEG segments represented different morphologies. The CL micelles containing approximately one mPEG segment can exhibit ‘echini’ morphology whereas the CL micelle with approximately three mPEG segments can form nanowires. The hydrolysis rate of the CL micelles is highly pH-dependent and is faster at mild acidic than normal physiological conditions. An antibiotic and/or a pH indicator loaded within the CL micelles can show a pH-dependent release behavior.

Other exemplary and non-limiting block copolymer micelles for pH-triggered delivery of the payload are synthesized and characterized as follows. The micelles are formed by the self-assembly of an amphiphilic diblock copolymer including a hydrophilic poly(ethylene glycol) (PEG) block and a hydrophobic polymethacrylate block (PEYM) bearing acid-labile ortho ester side-chains. The diblock copolymer is synthesized by atom transfer radical polymerization (ATRP) from a PEG macro-initiator to obtain well-defined polymer chain-length. The PEG-b-PEYM micelles can assume a stable core-shell structure in aqueous buffer at normal physiological pH with a low critical micelle concentration, which can be determined by proton NMR and pyrene fluorescence spectroscopy. The hydrolysis of the ortho ester side-chain at physiological pH can be minimal and can be much accelerated at mildly acidic pHs, as shown below.

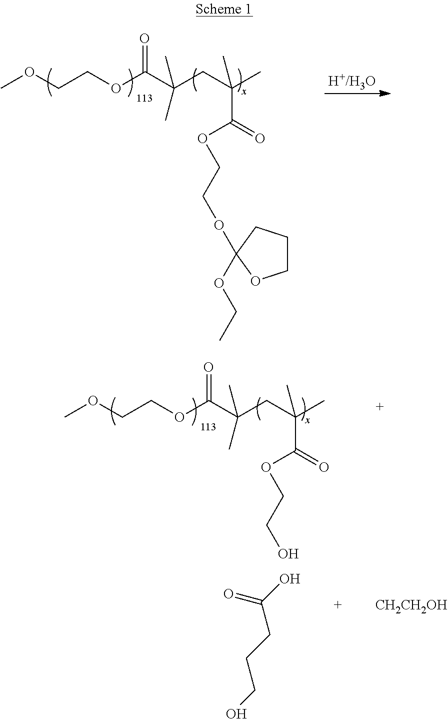

As will be apparent to the skilled artisan, the molecular weights and the structural features such as the orthoester, the alkyl groups, and such others shown above are merely illustrative and other such moieties, easily recognized by the skilled artisan, are also useful for preparing such micelles. Various payloads can be loaded within the micelles, for example, at about pH 7.4 and released at a much higher rate in response to slight acidification to, for example, about pH 5.

### Acid Degradable Microspheres and Nanospheres

In some embodiment, the components which degrade upon a change in pH, preferably upon a pH reduction, are microspheres or nanospheres. Microencapsulation packages liquids and solids in spherical or substantially spherical particles of micron size (microspheres) or nanometer size (nanospheres). For preparing microspheres and nanospheres and materials suitable for use in such micro- or nanospheres, see, for example, Edlund et al., Advances in Polymer Science, 157: 67-112 (incorporated herein in its entirety by reference). Microspheres/nanospheres over a wide size range, from hundreds of nanometers to hundreds of microns, can be prepared by modifying the processing conditions, as are well known to the skilled artisan.

One method of preparing microspheres is coacervation. Coacervation, or phase separation, involves the dissolution of the polymer in a liquid in which the insoluble core material to be encapsulated is suspended. Compositional changes of the system, e.g., addition of salts, or a pH or temperature change, subsequently bring about precipitation of the polymer. Particles manufactured by this method are capsular in structure. Coacervation may thus be used for the entrapment of liquids and oils.

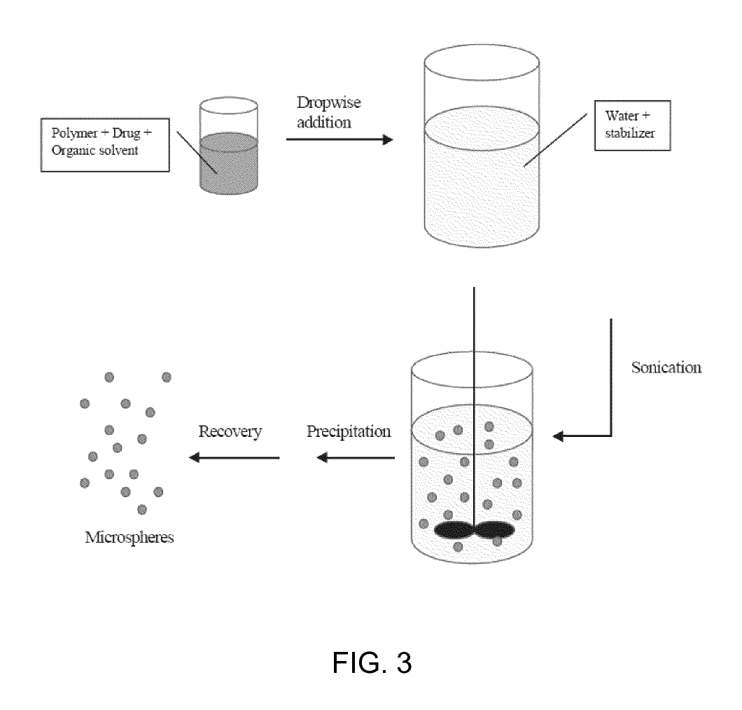

Oil-in-water (O/W) solvent-evaporation is another method that is schematically presented in FIG. 3. The antibiotic and the polymer are dissolved in a volatile organic solvent, typically methylene chloride. This oil phase is then dropwise added to a water phase, the latter containing a stabilizer such as polyvinyl alcohol or gelatin, under vigorous stirring. The faster the stirring, the smaller is the size of the particles obtained. The immiscibility of the two phases allows the formation of a stable emulsion. The role of the stabilizer, also referred to as emulsifier, is to prevent the droplets from coalescence and coagulation so that a stable emulsion is preserved. As the solvent is removed by evaporation, the polymer precipitates, a process sometimes facilitated by reduced pressure or by the addition of a nonsolvent. The hardened microspheres/nanospheres are subsequently separated from the aqueous phase, washed, and dried.

For the entrapment of water-soluble payloads (i.e., an antibiotic and/or a pH indicator), a double emulsion solvent evaporation technique, water-in-oil-in-water (W/O/W), can be employed. An aqueous solution of the payload is emulsified in an organic polymer solution. This water-in-oil emulsion is further emulsified into a continuous stabilized water phase. As the organic solvent is removed by evaporation, the polymer hardens and forms microspheres. The technique is also referred to as in-water drying.

Oil-in-oil (O/O) or water-in-oil-in-oil (W/O/O) solvent-removal techniques are also useful for preparing microspheres and/or nanopsheres. The use of oil as the continuous phase prevents the payload from partitioning out during processing. The (O/O) method is quite similar to the (O/W) technique but involves the dissolution of a polymer and the payload in an organic solvent, typically methylene chloride, and the emulsification of this organic phase in a second stabilized oil phase. When the (W/O/O) technique is employed, an aqueous solution of the payload is first emulsified in the organic polymer solution before the emulsion is added to the stabilized oil phase. Vegetable oils are preferred, since they are hydrophobic and edible. A stable emulsion is formed under vigorous stirring. The particles are formed as the organic solvent is extracted into the oil phase. The (W/O/O) technique is also termed in-oil drying.

Other techniques for preparing nano-microspheres include spray drying and hot-melt techniques. A polymer solution is sprayed into a heated chamber, which permits solvent evaporation and polymer precipitation. The method offers the advantage of allowing considerable control of the particle size. In this process the payload must withstand high temperatures without loss of activity. A microencapsulation process based on fluidized bed coating can also be used. This process involves the dissolution of the payload and the polymer in a mutual solvent. Capsules are formed as this solution is processed through a Wurster air suspension coater apparatus.

### Acid Degradable Polymers

A variety of acid hydrolyzable polymers are useful in the acid degradable components useful in this invention. In some embodiments, such polymers include a plurality of orthoesters. Such non-limiting and exemplary polymers are described above including in Yuan et al., (supra), and Tang et al., (supra, see also Scheme 1 above) and in Scheme 2 below.

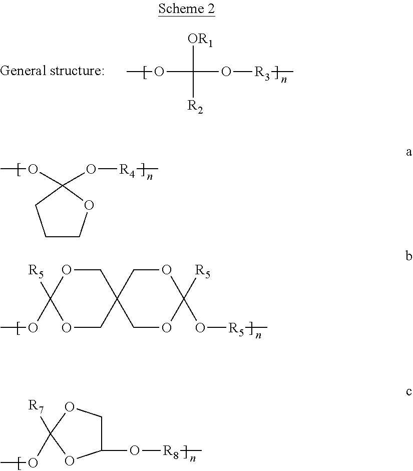

Certain exemplary and non-limiting class of polymers include poly(orthoesters) (POEs, see Scheme 2a) prepared by transesterification of a diol and diethoxytetrahydrofuran. See, for example, U.S. Pat. Nos. 4,079,038; 4,093,709; and 4,138,344; each of which is incorporated herein in its entirety by reference. Upon hydrolysis of these polymers the diol is regenerated and gamma-butyrolactone is formed; the latter readily hydrolyzes to hydroxybutyric acid. The formation of an acidic degradation product can create an acidic microclimate inside the device and can autocatalyze further degradation of the acid-labile POE.

Another class of exemplary and non-limiting polymer are based on the addition of diols to diketene acetals, typically 3,9-bis(ethylidene-2,4,8,10-tetraoxaspiro[5,5]undecane (R₅=CH₂CH₃ in Scheme 2b), using acid catalysts. These POEs degrade by hydrolysis to form the monomeric diol and pentaerythriol dipropionate. Mechanical and physical properties of these polymers can be manipulated by careful selection of the diol or by adding a mixture of diols. While diols result in linear POEs, cross-linked polymers may be obtained by adding alcohols of higher functionality. Dense cross-linked matrices can also be obtained by using this process. A cross-linked material is prepared in two steps. First, a prepolymer is prepared from two equivalents of diketene acetal and one equivalent of diol. A network is then formed by reaction the prepolymer with triols or a mixture of diols and triols. When the prepolymer is a viscous liquid, it can be mixed with the antibiotic and/or the pH indicator and cross-linked at rather low temperatures (e.g., about 40° C.). Another interesting property for drug delivery applications is that the degradation rate can be controlled.

Another exemplary class of POEs are prepared by the reaction between a triol and an alkyl orthoacetate (Scheme 2c). Depending on the triol used, everything from a sticky, ointment-like polymer to a solid, rigid material can be prepared. The use of 1,2,6-hexanetriol can produce erodible polymers with highly flexible backbones. Their consistency at room temperature can be that of a viscous paste, allowing for the payload to be incorporated without the use of solvent or elevated temperatures.

Other exemplary ad non-limiting acid degradable polymers include acetal containing polylactide-polyethylene glycols (PBELA) and galactose modified PBELA (PGBELA).

In some embodiments, a liquid bandage or a film of this invention can include one or more polymers that can be water-based and therefore make the liquid bandage water permeable. One example of a water-based polymer is polyvinylpyrrolidone, which is commonly called polyvidone or povidone, and made from the monomer N-vinylpyrrolidone.

When a polymer is used as the biocompatible material, such as a hydrogel, in the composition 110, the polymer, in some embodiments, is crosslinked to form a mesh. Crosslinking of the polymer ensures that the payload is entrapped with the polymer mesh. In one aspect, the polymer mesh includes at least a crosslinker.

A crosslinker is a chemical compound having two or more polymerizable groups. In general, any crosslinking compound may be used, so long as the polymerizable groups on the crosslinker are capable of forming a crosslinked co-polymer between the enzymes and the at least one monomer unit under the conditions used to form the nanocomplex. Examples of crosslinkers include compounds having two vinyl, acryl, alkylacryl, or methacryl groups. Examples of specific crosslinkers having two acryl groups include N,N′-methylenebisacrylamide and glycerol dimethacrylate. The crosslinker can be degradable or non-degradable. Degradable crosslinkers, such as those that degrade at certain pH, and further facilitate release of the antibiotic from the pH degradable components by allowing fluid communication between the composition (110) and the wound.

Examples of crosslinkers which degrade at reduced pH include glycerol dimethacrylate, which is stable at physiological pH (about 7.4), but hydrolyzes at lower pH (about 5.5). Further specific examples of degradable crosslinking groups include N,N′-methylenebis(acrylamide), 1,4-bis(acryloyl)piperazine, ethylene glycol diacrylate, N,N′-(1,2-dihydroxy-ethylene)bisacrylamide, and poly(ethylene glycol)diacrylate. Other examples of degradable crosslinkers include acetal crosslinkers described in U.S. Pat. No. 7,056,901, which is incorporated by reference in its entirety. Examples of non-degradable crosslinking groups include N,N′-bis(acryloyl)cystamine, glycerol dimethacrylate, bis[2-(methacryloyloxy)ethyl]phosphate, and bisacryloylated polypeptide.

These and other polymers are useful in the compositions and devices of this invention for pH sensitive release of antibiotics and/or identification of incipient infection. In certain embodiments, the microspheres, nanospheres, and matrices useful in this invention comprises use one or more of these polymers.

### Acid Degradable Hydrogels

In some embodiments, the composition is hydrated, and for example includes a hydrogel. In some embodiments, the components which degrade upon a change in pH are hydrogels and/or the corresponding xerogels.

In some aspects, the composition is non-hydrated. A non-limiting example of non-hydrated material is xerogel. In some embodiments, the components which degrade upon a change in pH are xerogels. When using a non-hydrated material, in one aspect, the outer layer (100) extends beyond the wound caring portion (130) at all directions. In this respect, the outer layer provides a complete seal of the wound site when the wound site is covered by the would caring portion. In the event blood or other types of tissue fluid comes out the wound, it is absorbed by the non-hydrated material in the composition. By virtue of the pressure applied to the wound site due to the seal in particular when the outer layer includes an elastic material, the device of this invention also helps stop bleeding at the wound site. Further, the water content of the blood or tissue fluid also serves as the basis for the CO₂ to form carbonic acid, which in turn activates the pH sensitive, preferably acid degradable components, entrapped in the device.

Suitable hydrogels are useful as matrices and as microspheres, nanospheres, and the like. See, for example, European Polymer Journal, 45 (6): 1689-97 and Macromolecular Bioscience, 4 (0): 957-62 (each of which is incorporated herein in its entirety by reference).

Certain exemplary and non-limiting acid degradable sugar based hydrogels are synthesized using a commercially available acid sensitive cross-linker, 3,9-divinyl-2,4,8,10-tetraoxaspiro-[5,5]-undecane. The monomers used for polymerization are N-isopropylacrylamide (NIPAM) and d-gluconamidoethyl methacrylate (GAMA), which when polymerized in the presence of the acid degradable cross-linker yield hydrogels that can swell and degrade under acidic conditions, making them suitable for antibiotic and/or pH indicator delivery. The hydrogels are synthesized using either a photo-initiator, Irgacure-2959 or a conventional initiator, potassium persulfate. The swelling capacity and antibiotic and/or pH indicator release from the hydrogels as a function of pH is tested by methods well known to the skilled artisan. The antibiotic and/or pH indicator release from the hydrogels can depend on the degree of cross-linking and the pH of the environment.

Other exemplary and non-limiting hydrogels include those based on di-acrylated Pluronic F-127 tri-block copolymer, prepared for example, by a photopolymerization method.

### Acid Degradable Matrices

In some embodiments, the component which degrades upon a change in pH, and is preferably acid degradable, is a matrix. The antibiotic and/or the pH indicator resides within the matrix. Various polymers and other materials suitable for use in other components which degrade upon a change in pH are useful in the matrices of this invention.

### Determining Absorption of Electromagnetic Radiation

Various methods for determining the absorption of the electromagnetic radiation by the byproduct or the derivative thereof or is well known to the skilled artisan, including, but not limited to, transmission and reflection based methods, which are applicable for determining IR and UV-visible absorption. Reflection based methods are suitable in some embodiments to determine the absorption from a bandage, a wound dressing, and such other coatings covering a wound. The signal to noise ratio in such detection can be improved, as is well known to the skilled artisan, following Fourier Transform (FT) techniques. Non-limiting examples of reflection based FT-IR methods for determining absorption include attenuated total reflection, specular reflection, and diffuse reflection methods.

### Uses

Compositions and devices of this invention can be used for wound caring, in particular a wound site (scrap, cut, catheter insertion site, etc.) that has bodily fluids which are potentially subject to infection. In use, the composition or device of this invention is affixed to the wound site, either with an external dressing, or via the adhesive surface on the device, or any other means. The composition or device keeps dirt and microorganisms out of the wound site. When infection occurs, the composition or device releases it antibiotic content which is used to treat infection.

It is to be understood that while the invention has been described in conjunction with the above embodiments, that the foregoing description and examples are intended to illustrate and not limit the scope of the invention. Other aspects, advantages and modifications within the scope of the invention will be apparent to those skilled in the art to which the invention pertains.

1. A method of detecting presence or absence of an incipient infection at a wound, the method comprising:
(i) contacting the wound with a wound dressing, the wound dressing permitting the accumulation of microbial byproducts or derivatives thereof;
(ii) measuring the change of an electromagnetic radiation absorption band of the byproduct or the derivative thereof; and
(iii) correlating the change of the electromagnetic radiation absorption band with the presence or absence of the incipient infection.

2. The method of claim 1, wherein the electromagnetic radiation is infra red (IR) radiation, or ultra violet (UV)-visible radiation.

3. The method of claim 1, wherein the wound dressing comprises carboxyl groups.

4. The method of claim 3, wherein the IR absorption of carboxyl groups or carboxylate anions corresponding to the carboxyl groups is determined.

5. The method of claim 1, wherein the wound dressing comprises a liposome comprising fatty acids, or wherein the composition comprises a poly caroboxylic acid polymer.

6. A topical pH sensitive composition for wound caring which comprises an antibiotic and a pH sensitive component, such that when administered topically the antibiotic is released upon a formation of an incipient infection at the wound.

7. A topical pH sensitive composition for wound caring which comprises an antibiotic, a pH indicator, and a pH sensitive component, such that when administered topically the antibiotic is released upon a formation of an incipient infection at the wound.

8. A topical pH sensitive composition for detection of an incipient infection at a wound, which composition comprises a pH indicator, and a pH sensitive component, such that when the composition is applied to the wound, the formation of the incipient infection at the wound provides a visible color change of the composition.

9. A topical wound caring device comprising:
a) a water-impermeable and hydronium ion impermeable outer layer having a top surface and a bottom surface;
b) an inner layer having a top surface and a bottom surface, the top surface of said inner layer affixed to at least a portion of the bottom surface of said outer layer, wherein said inner layer comprises (i) a water permeable, hydronium ion permeable and biocompatible material and (ii) a pH-sensitive component, which component comprises a therapeutic amount of at least an antibiotic,
wherein the bottom surface of said inner layer is capable to contact said wound when in use.

10. A topical wound caring device comprising:
a) a water-impermeable and hydronium ion impermeable outer layer having a top surface and a bottom surface;
b) an inner layer having a top surface and a bottom surface, the top surface of said inner layer affixed to at least a portion of the bottom surface of said outer layer, wherein said inner layer comprises (i) a water permeable, hydronium ion permeable and biocompatible material and (ii) a pH-sensitive component, which component comprises a therapeutic amount of at least an antibiotic and at least a pH indicator in an amount effective for detecting a color change thereby evidencing a change in pH,
wherein the bottom surface of said inner layer is capable to contact said wound when in use.

11. An incipient infection detection device comprising:
a) a water-impermeable and hydronium ion impermeable outer layer having a top surface and a bottom surface;
b) an inner layer having a top surface and a bottom surface, the top surface of said inner layer affixed to at least a portion of the bottom surface of said outer layer, wherein said inner layer comprises (i) a water permeable, hydronium ion permeable and biocompatible material and (ii) a pH-sensitive component, which component comprises at least a pH indicator in an amount effective for detecting a color change thereby evidencing a change in pH,
wherein the bottom surface of said inner layer is capable to contact said wound when in use.

12. The device of any one of claims 9-11, wherein said outer layer extends beyond said inner layer.

13. The device of any one of claims 9-11, wherein said outer layer further comprises an adhesive surface on the bottom surface.

14. The device of any one of claims 9-13, wherein said water permeable, hydronium ion permeable and biocompatible material is selected from the group consisting of a hydrogel, woven cotton, and woven cellulose.

15. The device of any one of claims 9-14, wherein said antibiotic is selected from the group consisting of ampicillin, ampicillin and sulbactam, augmentin, bacitracin, cefazolin, cefotaxime, cefotetan, cefoxitin, ceftriaxone, cephalexin, ciprofloxacin, dicloxacillin, duricef, erythromycin, imipenem, metronidazole, piperacillin and tazobactam, polymyxin, ticarcillin and clavulanic acid, and combinations thereof.

16. The device of anyone of claims 9-15, wherein said pH sensitive component comprises an acid degradable liposome, an acid degradable micelle, an acid degradable hydrogel or xerogel, or an acid degradable matrix.

17. The device of claim 16, wherein said liposome comprises one or more of unsaturated phosphatidylethanolamines combined with mildly acidic amphiphiles, caged lipid derivatives, pH-sensitive peptides, or pH-titratable polymers.
